(19) United States
(12) Patent Application Publication
(10) Pub. No.: US 2008/0193550 A1
(43) Pub. Date: Aug. 14, 2008

(54) Cla-Enriched Milkfat and Uses Thereof

(75) Inventors: Alastair Kenneth Hugh MacGibbon, Palmerston North (NZ); Geoffrey Wayne Krissansen, Auckland (NZ); Rupinder Kaur Kanwar, Geelong (AU); Peter Nigel Black, Auckland (NZ)

Correspondence Address: KNOBBE MARTENS OLSON & BEAR LLP, 2040 MAIN STREET, FOURTEENTH FLOOR, IRVINE, CA, 92614, US

(73) Assignees: FONTERRA CORPORATE RESEARCH AND DEVELOPMENT LIMITED, AUCKLAND (NZ); AUCKLAND UNISERVICES LIMITED, AUCKLAND (NZ)

(21) Appl. No.: 11/596,196

(22) PCT Filed: May 11, 2005
(86) PCT No.: PCT/NZ05/00096

§ 371 Date: Mar. 25, 2008

(30) Foreign Application Priority Data
May 11, 2004 (NZ) 532872

(51) Int. Cl. A61K 35/20; A61P 37/08; A61P 11/06

(52) U.S. Cl. 424/535

(57) ABSTRACT

The present invention relates to use of c-9, t-11 CLA or a salt, ester or precursor thereof or CLA-enriched milk fat comprising milk fat enriched with c-9, t-11 CLA or a salt, ester or precursor thereof for treating or preventing conditions such as those associated with one or more of leukocyte infiltration, eosinophilia, IgE secretion, airway remodelling, bronchoconstriction and mucus hypersecretion. The invention also relates to a pharmaceutical composition comprising CLA-enriched milk fat.

### FIELD OF THE INVENTION

The present invention relates to use of the c-9, t-11 isomer of conjugated linoleic acid (CLA) to treat or prevent conditions associated with one or more of leukocyte infiltration, eosinophilia, airway remodelling and bronchoconstriction. The invention also relates to a CLA-enriched milk fat composition and its use in methods of treating or preventing conditions associated with one or more of leukocyte infiltration, eosinophilia, IgE secretion, airway remodelling, bronchoconstriction and mucus hypersecretion.

### BACKGROUND

Persons with atopy have a genetic predisposition to produce IgE antibodies against comrnon environmental allergens, and often suffer from one or more atopic diseases including allergic rhinitis, asthma, and atopic eczema (1). Atopic individuals have an exaggerated response to allergen characterized by elevated levels of IgE antibodies, and their T cells respond to allergen by producing type 2 helper (Th2) cytokines, including interleukin-4 (IL-4), IL-5, IL-9 and IL-13 rather than the type 1 helper (Th1) cytokines IL-2 and interferon-gamma (IFN-gamma) that typify the normal response.

Exposure of a person with atopy to allergen can lead to an immediate hypersensitivity reaction in which a complex of allergen, IgE, and FcεRI on the surface of mast cells triggers the release of histamine, tryptase, and the lipid mediators leukotrienes, prostaglandins, and platelet-activating factor. The leukotrienes C4, D4, and E4 cause the contraction of smooth muscles, vasodilatation, increased vascular permeability, and hypersecretion of mucus. Tryptase activates a signalling pathway that leads to the upregulation of cell adhesion molecules on endothelial and epithelial cells that selectively attract eosinophils and basophils. In the subsequent late-phase reaction, eosinophils and neutrophils accumulate in the lung, followed by CD4+ T cells. Late-phase reactions can be induced in the absence of immediate hypersensitivity indicating T cells alone are sufficient to initiate narrowing of the airways in patients with allergic asthma.

Increased numbers of eosinophils is a hallmark of allergic disease, and eosinophils are enriched up to 100-fold in the airways of asthmatic subjects. A recent review reported that there is a broad correlation between the degree of eosinophilia and disease severity. Eosinophils are a characteristic feature of seasonal and perennial rhinitis (2) and nasal polyposis (3). There are increased numbers of eosinophils in atopic dermatitis, and deposition of eosinophil basic proteins in the affected skin (4). Degranulating eosinophils can injure mucosal surfaces by releasing toxic basic proteins, cysteinyl leukotrienes, and platelet activating factor which are thought to cause bronchospasm; and impair M2 muscarinic receptors responsible for controlling cholinergic responses. They have been proposed to play pathogenic roles in asthma, nasal polyposis, allergic rhinitis, and eosinophilic pneumonia (5,6).

Asthma attacks are triggered by the binding of inhaled allergens to IgE antibodies on the surfaces of sensitised mast cells in the lungs. Binding triggers mast cell degranulation and release of histamine and leukotrienes. These molecules cause the smooth muscle cells of the bronchi to contract, narrowing the lumen of the bronchi, attract inflammatory cells, especially eosinophils, and mediate mucus production. Existing medicines that are mast cell stabilisers inhibit immediate allergic responses but are not effective in treating chronic asthma. A medicine that inhibits mediator release from mast cells is unlikely to be an effective treatment for asthma unless it can be shown to have some other activity e.g. as a bronchodilator or inhibitor of eosinophilic inflammation.

Inhaled corticosteroids are now the recommended first-line therapy for asthma, as they improve lung function, decrease symptoms, reduce exacerbations, and can prevent more than half of all hospitalizations due to asthma (7). They are effective at reducing morbidity and mortality due to asthma, but they have to be regularly inhaled to remain effective. Inhaled corticosteroids are in some cases being prescribed for asthma at inappropriately high doses, with the potential to cause adverse effects such as osteoporosis, cataracts and adrenal suppression (8). A variety of therapeutic agents have been administered to asthma patients because of their steroid-sparing effect, including anti-IgE antibodies (9), leukotriene receptor antagonists (10), gold and methotrexate (11). Steroid-resistant asthma in which the patient derives reduced benefit from steroid use, is a serious medical challenge, and requires the delivery of non-steroidal anti-asthmatic drugs (12).

The Western lifestyle is believed to be a contributing factor to the risk of developing asthma. Diets have changed significantly since we led a more pastoral existence. Epidemiological studies have suggested a beneficial effect of consuming oily fish (13), however the results of intervention studies with fish oil has been inconsistent. A reduction in the levels of inflammatory mediators associated with asthma has been reported with dietary interventions such as administration of oils containing a combination of gamma-linolenic acid and eicosapentaenoic acid (EPA), normally derived from fish (14). Dietary supplementation with fish oil rich in EPA and docosahexaenoic acid (DHA) has been reported to be beneficial for children with bronchial asthma (15). A lipid extract from the New Zealand green-lipped mussel (Perna canaliculus) rich in the omega 3 fatty acids DHA and EPA reportedly decreased daytime wheeze, the concentration of exhaled H₂O₂, and increased morning peak expiratory flow in asthma patients (16). A number of other studies have not shown any benefit from treatment with fish oil (17).

A recent study investigated the relationship between food consumption and asthma symptoms in 2978 pre-school children followed prospectively. It reported that the frequent consumption of products containing milk fat was associated with a reduced risk of asthma symptoms (18). A number of other studies have suggested that consumption of dairy products can protect against the development of allergic sensitisation or atopic disease, and that conversely that polyunsaturated fat may be deleterious (19-22).

Milkfat contains a number of bioactive fatty acids. The most extensively studied fatty acid from milk is conjugated linoleic acid (CLA), which has been reported to exhibit a number of health benefits (23). The tracheae of guinea pigs fed synthetic CLA enriched in t-10, c-12 isomer for two weeks reportedly displayed reduced contraction to allergen, which corresponded with increased release of prostaglandin E2 (PGE2) (International Patent Application WO 97/32008). In contradiction, the same authors reported in two subsequent papers that feeding of an approximately equal mixture of synthetic cis-9, trans-11 and trans-10, cis-12 isomers of CLA reduced allergen-induced histamine and release of PGE2 from allergen sensitized guinea pig tracheae (24,25), but did not affect allergen-induced tracheal contractions (24).

Whilst the health benefits of synthetically prepared CLAs have been reported, there is a paucity of infonnation on the properties of naturally occurring CLAs in human and bovine milk. Bovine milk fat contains principally (75-90%) the c-9, t-11 isomer (26). CLA is produced naturally in the rumen as an intermediate in the biohydrogenation of dietary linoleic acid to stearic acid and in tissues by the action of the delta-9 desaturase enzyme on trans vaccenic acid (trans-11-octadecenoic acid). The second most prevalent CLA isomer in milk fat is the t-7, c-9 isomer, but it is present at about 10% of the level of c-9, t-11 isomer. The milk fat content of the t-10, c-12 isomer of CLA can be markedly increased under certain dietary situations, but is still less than 2% of the c-9, t-11 CLA content (27). Milk fat contains traces of many additional isomers of CLA.

An exhaustive analysis of the published data on the influence of synthetic seed-derived CLA on immune function reported that supplementation of the diet with CLA is not recommended (28). The synthetic c-9, t-11 CLA isomer appears relatively benign, whereas in contrast, the synthetic t-10, c-12 isomer has been shown to alter body fat mass, increase the fat content of several tissues, increase circulating insulin, and increase the saturated fatty acid content of adipose tissue and muscle (28). In addition, it has been reported to cause a dramatic enlargement of the liver with steatosis when fed to mice at 0.4% w/w for 4 weeks (29). t-10, c-12 CLA has also been shown to have deleterious effects in man (30). This latter study showed that t-10, c-12 CLA aggravated insulin resistance and increased CRP and 8-iso-prostane which is a marker of oxidative stress.

It would therefore be desirable to provide an improved or alternative means for treating or preventing conditions such as atopic conditions, eosinophilias and Th2 mediated conditions that overcomes or ameliorates problems associated with reported treatments or that at least provides the public with a useful choice.

### SUMMARY OF THE INVENTION

Accordingly, in one aspect the present invention provides use of c-9, t-11 CLA or a salt, ester or precursor thereof in the manufacture of a composition for treating or preventing a condition associated with one or more of leukocyte infiltration, eosinophilia, airway remodelling and bronchoconstriction. In one embodiment the condition is selected from the conditions listed below including atopic conditions, eosinophilias and Th2-mediated conditions. In one embodiment the condition is asthma.

In one embodiment the composition is substantially free of the t-10, c-12 CLA isomer.

In another aspect the present invention provides use of milk fat enriched with c-9, t-11 CLA or a salt, ester or precursor thereof in the manufacture of a composition for treating or preventing a condition associated with one or more of leukocyte infiltration, eosinophilia, IgE secretion, airway remodelling, bronchoconstriction and mucus hypersecretion. In one embodiment the condition is selected from the conditions listed below including atopic conditions, eosinophilias and Th2-mediated conditions. In one embodiment the condition is asthma.

In one embodiment the CLA-enriched milk fat comprises at least about 2, 4, 6, 8, 10, 15, 20, 25, 30, 35, 40, 45 or 50% by weight of c-9, t-11 CLA or a salt, ester or precursor thereof and useful ranges may be selected between any of these forgoing values (e.g. from about 4% to about 7%). Preferably the milk fat comprises at least about 2% c-9, t-11 CLA by weight, preferably about 2 to 10% c-9, t-11 CLA by weight, more preferably about 4 to 7% c-9, t-11 CLA by weight and most preferably about 5% c-9, t-11 CLA by weight.

In one embodiment the milk fat comprises CLA isomers which comprise at least about 50, 55, 60, 65, 70, 75, 80, 85, 90, 95 or 99% c-9, t-11 CLA by weight or a salt, ester or precursor thereof and useful ranges may be selected between any of these forgoing values (e.g. from about 80% to about 95%). Preferably the milk fat includes CLA isomers comprising at least about 50% c-9, t-11 CLA by weight, preferably about 70 to 90% c-9, t-11 CLA by weight, preferably about 70 to 80% c-9, t-11 CLA by weight.

In one embodiment the c-9, t-11 CLA is selected from c-9, t-11 CLA derived from a natural source; synthetic c-9, t-11 CLA; c-9, t-11 CLA in free fatty acid form; c-9, t-11 CLA bound to glycerol, a monoglyceride or a diglyceride; c-9, t-11 CLA in esterified form; or mixtures thereof.

In one embodiment the milk fat is substantially free of the t-10, c-12 CLA isomer or the milk fat otherwise has a fatty acid profile that corresponds substantially to the fatty acid profile of normal milk fat. In one embodiment the milk fat composition comprises the t-10, c-12 CLA isomer at a level that is no greater than the level of the t-10, c-12 CLA isomer found in normal milk fat.

In one embodiment the composition further comprises one or more constituents (such as antioxidants) which prevent or reduce degradation of the composition during storage or after administration.

In one embodiment the milk fat is produced by enhancing natural levels of CLA in milk by feeding a milk producing mammal with a diet enriched in at least one fatty acid (e.g. linoleic acid).

In another embodiment the milk fat composition of the invention is prepared by combining a source of c-9, t-11 CLA or a salt, ester or precursor thereof with milk fat.

In one embodiment the composition is formulated as a food, drink, food additive, drink additive, dietary supplement, nutritional product, medicament, pharmaceutical or neutraceutical. Preferably, the composition is formulated as a powder, liquid, food bar, spread, sauce, ointment, tablet or capsule.

In one embodiment the composition is formulated for oral, nasal, topical, subcutaneous, intramuscular, intravenous or parenteral administration.

In one embodiment the composition is formulated for ingestion, inhalation or topical application. Where the composition is formulated for inhalation, preferably it is formulated as an inhalable powder, solution or aerosol. Where the composition is formulated for topical application, preferably it is formulated as an ointment, cream or lotion.

In one embodiment the use is for treating or preventing a condition associated with one or more of leukocyte infiltration, eosinophilia, IgE secretion, airway remodelling, bronchoconstriction and mucus hypersecretion with steroid sparing effect. Preferably the condition is a steroid-dependent condition including corticosteroid dependent asthma, severe eczema and eosinophilic disorders including eosinophilic gastroenteritis, eosinophilic pneumonia and hyper-eosinophilic syndrome.

Another aspect of the present invention provides a pharmaceutical composition comprising milk fat enriched with c-9, t-11 CLA or a salt, ester or precursor thereof as described above, and a pharmaceutically acceptable carrier.

In one embodiment a pharmaceutical composition of the invention is formulated for oral, nasal, topical, subcutaneous, intramuscular or intravenous administration. In another embodiment a pharmaceutical composition of the invention is formulated for ingestion, inhalation or topical application. In yet another embodiment a pharmaceutical composition of the invention is formulated as an inhalable powder, inhalable solution or aerosol.

Another aspect of the present invention provides a method for treating or preventing a condition associated with one or more of leukocyte infiltration, eosinophilia, airway remodelling and bronchoconstriction, including those listed below, comprising administering c-9, t-11 CLA or a salt, ester or precursor thereof to a subject in need thereof.

Another aspect of the present invention provides a method for treating or preventing a condition associated with one or more of leukocyte infiltration, eosinophilia, IgE secretion, airway remodelling, bronchoconstriction and mucus hypersecretion, including but not limited to those listed below, comprising administering milk fat enriched with c-9, t-11 CLA or a salt, ester or precursor thereof, as described above to a subject in need thereof.

In one embodiment the condition is asthma.

In one embodiment the condition is an atopic condition. In another embodiment the condition is an allergy. In yet another embodiment the condition is an eosinophilia. In still another embodiment the condition is a Th2 mediated condition.

In one embodiment the condition is selected from allergic rhinitis, hay fever, atopic rhinoconjunctivitis, urticaria, asthma and atopic eczema.

In one embodiment the condition is selected from contact dermatitis, eczema, hives (urticaria), allergic conjunctivitis, hay fever, allergic rhinitis, airborne allergies including tree (e.g. birch pollen), weed (e.g. ragweed), and grass pollen allergies, latex allergies, food allergies (e.g. peanut, shellfish, milk protein), drug allergies (e.g. to penicillin), insect sting allergies (e.g. honeybee allergies, wasp allergies, hornet allergies, yellow jacket allergies, fire ant allergies), mold allergies (e.g. to alternaria, cladosporium, aspergillus, penicillium, helminthosporium, epicoccum, fusarium, mucor, rhizopus, and aureobasidium), dust mite allergies, animal allergies (e.g. household pets such as cats and dogs), allergic bronchopulmonary aspergillosis, occupational asthma, and episodic angioedema with eosinophilia.

In one embodiment the condition is selected from airway, lung, blood and skin eosinophilia. In another embodiment, the eosinophilia is selected from eosinophilic ascites, eosinophilic cellulitis, eosinophilic fasciitis, eosinophilic gastroenteritis, coeliac disease, allergic colitis, eosinophilic esophagitis, eosinophilic pancreatitis, eosinophilic pneumonias, bronchiectasis, eosinophilic synovitis, nasal eosinophilia, tropical pulmonary eosinophilia, Churg Strauss syndrome, pulmonary eosinophilia, idiopathic hyper-eosinophilic syndrome, inflammatory bowel disease, eosinophilic cholangitis, eosinophilic leukaemia and other eosinophilic cancers, familial (hereditary eosinophilia), eosinophilic granuloma, sarcoidosis, eosinophilia-myalgia syndrome, cystic fibrosis, nasal polyposis, eosinophil meningitis, Wegener's granulomatosis, polyarteritis nodosa, rheumatoid arthritis, pemphigus vulgaris, bullous pemphigoid, dermatitis herpetiformis, erythema multiforme, eosinophilic cellulites, parasitic infections (Ascaris Toxocara canis, Filariasis, Anchylostomiasis, Trichinosis, Strongvloidiasis, Fascioliasis, Schistosomiasis).

In one embodiment the condition is selected from Th2 mediated asthma, allergies, eczema, microbial or parasite infection, and autoimmune diseases including ulcerative colitis.

Another aspect of the present invention provides a method for treating or preventing a condition associated with one or more of leukocyte infiltration, eosinophilia, IgE secretion, airway remodelling, bronchoconstriction and mucus hypersecretion with steroid sparing effect comprising administering c-9, t-11 CLA or a salt, ester or precursor thereof or milk fat enriched with c-9, t-11 CLA or a salt, ester or precursor thereof to a subject in need thereof. Preferably the condition is a steroid-dependent condition including corticosteroid dependent asthma, severe eczema and eosinophilic disorders including eosinophilic gastroenteritis, eosinophilic pneumonia and hyper-eosinophilic syndrome.

The entire disclosures of all applications, patents and publications, cited above and below, if any, are hereby incorporated by reference.

The term “comprising” as used in this specification and the claims means “consisting at least in part of”. When interpreting statements in this specification and the claims which include that term, the features, prefaced by that term in each statement or claim, all need to be present but other features can also be present.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### BRIEF DESCRIPTION OF THE DRAWINGS

FIG. 1A is a graph showing the mean number of BAL cells (±SEM) from healthy mice fed a control chow diet (similar results were obtained with healthy mice fed an AIN93G control diet) and OVA challenged mice fed experimental diets as described in Table 1 (n=5 to 6 mice per group).

FIG. 1B is a graph showing the mean cell counts (±SEM) of cell types present in BAL from healthy mice fed a control diet and OVA challenged mice fed experimental diets as described in Table 1 (n=5 to 6 mice per group).

FIGS. 2A and 2B are graphs showing mean levels (±SEM) of allergen-specific IgE and IgG1 responses in healthy mice fed a control diet and OVA challenged mice fed experimental diets as described in Table 1 (n=5 to 6 mice per group).

FIG. 3A is a graph showing the mean number of BAL cells (±SEM) from OVA challenged mice fed experimental diets as described in Table 2 (n=5 to 6 mice per group).

FIG. 3B is a graph showing the mean cell counts (±SEM) of cell types present in BAL from OVA challenged mice fed experimental diets as described in Table 2 (n=5 to 6 mice per group).

FIG. 4 is a graph showing the total number of BAL cells as individual data, and means from OVA challenged mice fed experimental diets as described in Table 3 (n=4 to 6 mice per group).

FIG. 5 is a graph showing the number of each cell type present in the BAL cells as individual data, and means from OVA challenged mice fed experimental diets as described in Table 3 (n=4 to 6 mice per group).

### DETAILED DESCRIPTION OF THE INVENTION

As shown in the Examples, a CLA-enriched milk fat composition suppressed the development of OVA-induced airway inflammation in a mouse model of allergen (ovalbumin)-induced asthma. In contrast, normal milk fat and normal milk fat spiked with a synthetic CLA product derived from safflower oil (“syn-CLA”) had no detectable effect. Dietary administration of c-9, t-11 CLA in free fatty acid or glyceride form was found to reduce lung inflammation but to a lesser degree than CLA-enriched milk fat.

The term “normal milk fat” is intended to mean typical mammalian milk fat. For example, milk fat produced by New Zealand pasture fed cows. A compositional analysis of typical New Zealand milk fat and anhydrous milk fat (AMP) is presented in Tables 1 and 2. The composition of New Zealand milk fat may vary from season to season as known in the art (See for example, MacGibbon A K H, Van der Does Y E H, Fong B Y, Robinson N P, Thomson N A, “Variations in the CLA content of New Zealand Milkfat”, Australian Journal of Dairy Technology (2001), 56(2), 158).

The terms “CLA-enriched milk fat” and “milk fat enriched with c-9, t-11 CLA” are intended to mean milk fat that comprises a higher level of c-9, t-11 CLA or a salt, ester or precursor thereof than normal milk fat. CLA-enriched milk fat may prepared by known techniques including but not limited to supplementary free fatty acid feeding of pasture fed cows (32). CLA-enriched milk fat may also be prepared by supplementing milk fat with CLA. Milk fat for use according to the invention may in one embodiment be sheep, goat, pig, mouse, water buffalo, camel, yak, horse, donkey, llama, bovine or human milk fat. Preferably the milk fat is bovine milk fat.

In one embodiment the CLA-enriched milk fat comprises at least about 2, 4, 6, 8, 10, 15, 20, 25, 30, 35, 40, 45 or 50% by weight of c-9, t-11 CLA or a salt, ester or precursor thereof and useful ranges may be selected between any of these forgoing values (e.g. from about 4% to about 7%). Preferably the CLA-enriched milk fat comprises at least about 2% c-9, t-11 CLA by weight, preferably about 2 to 10% c-9, t-11 CLA by weight, more preferably about 4 to 7% c-9, t-11 CLA by weight and most preferably about 5% c-9, t-11 CLA by weight.

In one embodiment the CLA-enriched milk fat comprises CLA isomers which comprise at least about 50, 55, 60, 65, 70, 75, 80, 85, 90, 95 or 99% by weight c-9, t-11 CLA or a salt, ester or precursor thereof and useful ranges may be selected between any of these forgoing values (e.g. from about 80% to about 95%). Preferably the CLA-enriched milk fat includes CLA isomers comprising at least about 50% c-9, t-11 CLA by weight, preferably about 70 to 80% c-9, t-11 CLA by weight.

In one embodiment, the c-9, t-11 CLA isomer may be included in a composition of the invention in free fatty acid form. In another embodiment the CLA may be in an esterified form, including but not limited to methyl, ethyl and propyl esters. In another embodiment the CLA may be in a salt form, including but not limited to sodium salts and zinc salts. In a further embodiment, one or more c-9, t-11 CLA molecules may be bound to a polyol such as glycerol or sphingosine, with or without other fatty acids, to form mono-, di- or tri-glycerides for example. In yet another embodiment, mixtures of these forms of c-9, t-11 CLA may be included within a composition of the invention. In another embodiment a precursor of c-9, t-11 CLA may be provided including but not limited to vaccenic acid (trans-11-octadecenoic acid).

Appropriate levels of the c-9, t-11 CLA isomer or a salt, ester or precursor thereof may be determined, obtained and provided by a skilled worker with regard to that skill and to the teaching of the present application.

Referring to the Examples, CLA-enriched milk fat reduced allergen-specific IgE and IgG1 levels by 30 to 55%, and 45 to 48%, respectively, compared with normal milk fat. CLA-enriched milk fat inhibited lung eosinophilia and lymphocytosis, whereas milk fat and syn-CLA-spiked normal milk fat had no discernible affect. CLA-enriched milk fat inhibited goblet cell metaplasia and the overproduction of airway mucus (features of airway remodelling), whereas normal milk fat and syn-CLA-spiked normal milk fat had no discernible affect. The airways of mice fed CLA-enriched milk fat were open, whereas the airways of mice fed normal milk fat and syn-CLA-spiked normal milk fat were occluded with mucin and constricted.

Mice fed the highest doses of syn-CLA-spiked normal milk fat, where the synthetic CLA isomers represented 5.3% of the diet, developed fatty liver disease. The CLA-enriched milk fat composition had no such effect, and visual inspection of other organs did not reveal any toxic side-effects, even at the highest doses.

Syn-CLA separated into its two major components, namely synthetic c-9, t-11 (syn-9,11 CLA) and t-10, c-12 (syn-10,12 CLA) CLA isomers, revealed syn-9,11 CLA inhibited lung eosinophilia and lymphocytosis, whereas syn-10,12 CLA had no discernible affect.

CLA-eniched milk fat diminished allergen-specific Ig reponses compared to normal milk fat and syn-CLA whereas fiee fatty acid and triglyceride forms of CLA isomers had no significant effect.

Thus, the CLA-enriched milk fat composition described herein is able to reduce one or more of leukocyte infiltration, eosinophilia, IgE secretion, airway remodelling, bronchoconstriction and mucus hypersecretion, and so is useful as a therapy for conditions where reducing one or more of these states is beneficial. Such conditions include atopic conditions, allergies, eosinophilias and Th2-mediated conditions.

Accordingly, in one aspect the present invention relates to use of c-9, t-11 CLA or a salt, ester or precursor thereof in the manufacture of a composition for treating or preventing a condition associated with one or more of leukocyte infiltration, eosinophilia, airway remodelling and bronchoconstriction.

In another aspect the present invention relates to use of milk fat enriched with c-9, t-11 CLA or a salt, ester or precursor thereof in the manufacture of a composition for treating or preventing a condition associated with one or more of leukocyte infiltration, eosinophilia, IgE secretion, airway remodelling, bronchoconstriction and mucus hypersecretion.

The c-9, t-11 CLA may be synthetic, derived from a natural source, or mixtures thereof. Natural sources of c-9, t-11 CLA are described by Chin et al (31). In one embodiment where the c-9, t-11 CLA is synthetic CLA, the CLA includes CLA that is chemically modified to improve potency, stability, transport and half-life.

Sunflower and safflower seed oils, containing approximately 65% and 76% linoleic acid respectively, are currently used as raw material for CLA production. Optimal conditions used in commercial scale production results in approximately equal amounts of the isomers c-9, t-11 and t-10, c-12. A safflower based product can thus contain approximately 36% each of c-9, t-11 and t-10, c-12 isomers. Minor peaks are include the cis, cis and trans, trans isomers of 9,11 and 10,12 CLA, each around 0.5 to 1%. Traces of c-11, t-13 (which is formed from heating the t-10, c-12 isomer) and t-8, c-10 (from heating of the c-9, t-11 isomer) may also be present.

A composition for use according to the invention may optionally further comprise at least one antioxidant or other agent able to prevent degradation of the c-9, t-11 CLA or salt, ester or precursor thereof.

In one embodiment, the milk fat or composition for use according to the invention is substantially free of the t-10, c-12 CLA isomer which may neutralize the protective affect of the c-9, t-11 CLA isomer if it is present in high levels compared to the amount of the c-9, t-11 CLA isomer present. Preferably, to obtain maximal benefits, patients should minimise their use of non-animal commercially-prepared synthetic mixed CLA isomers and of other food sources containing relatively high levels of the t-10, c-12 CLA isomer.

Without wishing to be bound by theory, it is possible that other CLA isomers (apart from the 10, 12 isomers) and that one or more trans fatty acids (in free fatty acid or glyceride form), such as C10 to C20 fatty acids, may be contributing to the activity of the milk fat composition of the invention. Other potentially active CLA isomers include one or more of the t-9, c-11; c-8, t-10; c-8, c-10; c-9, c-11; c-11, c-13; t-11, t-13; or t-9, t-11 CLA isomers.

In one embodiment, the milk fat composition otherwise has a fatty acid profile that corresponds substantially to the fatty acid profile of normal milk fat.

In one embodiment the CLA-enriched milk fat comprises increased levels of vaccenic acid. Preferably the CLA-enriched milk fat comprises at least about 5, 10, 15, 20, 25, 30 or 35% by weight vaccenic acid.

In one embodiment the CLA-enriched milk fat comprises decreased levels of c16:0.

In one embodiment, the milk fat composition comprises normal milk fat where the fatty acid profile is altered due to seasonal variation or to variations due to dietary supplementation, as known in the art, and in a preferred embodiment includes milk fat having the fatty acid profile of the CLA-enriched milk fat set out in Table 2.

Optionally, the milk fat composition further comprises one or more constituents (such as antioxidants) which prevent or reduce degradation of the composition during storage or after administration.

In one embodiment, the milk fat composition comprises the t-10, c-12 CLA isomer at a level that is no greater than the level of the t-10, c-12 CLA isomer found in normal milk fat. Thus, for the purposes of this embodiment, the milk fat composition is substantially fiee of the t-10, c-12 CLA isomer, as discussed above, except for the t-10, c-12 CLA that is naturally present in the milk fat itself.

In one embodiment, the milk fat composition is produced by enhancing natural levels of CLA in milk by feeding a milk producing mammal with a diet enriched in at least one fatty acid (e.g. linoleic acid). See for example the method described by Harfoot et al (32).

In another embodiment, the milk fat composition of the invention is prepared by combining a source of c-9, t-11 CLA with milk fat.

A composition useful herein may be formulated as a food, drink, food additive, drink additive, dietary supplement, nutritional product, neutraceutical, medicament or pharmaceutical. Preferably, a composition of the invention is formulated as a powder, liquid, food bar, spread, sauce, ointment, tablet or capsule. Appropriate formulations may be prepared by an art skilled worker with regard to that skill and the teaching of this specification.

The present invention also provides a pharmaceutical composition comprising a CLA-enriched milk fat as described above and a pharmaceutically acceptable carrier.

Another aspect of the invention provides a method for treating or preventing conditions associated with one or more of leukocyte infiltration, eosinophilia, IgE secretion, airway remodelling, bronchoconstriction and mucus hypersecretion, including those listed below, comprising administering c-9, t-11 CLA or a salt, ester or precursor thereof or milk fat enriched with c-9, t-11 CLA or a salt, ester or precursor thereof to a subject in need thereof.

Thus, one aspect of the invention provides a method of treating or preventing an atopic condition comprising administering c-9, t-11 CLA or a salt, ester or precursor thereof or milk fat enriched with c-9, t-11 CLA or a salt, ester or precursor thereof to a subject in need thereof. In one embodiment, the atopic condition is selected from allergic rhinitis, hay fever, atopic rhinoconjunctivitis, urticaria, asthma and atopic eczema.

A “subject” in accordance with the invention is an animal, preferably a mammal, more preferably a mammalian companion animal or human. Preferred companion animals include cats, dogs and horses.

Another aspect of the invention provides a method of treating or preventing an allergy comprising administering c-9, t-11 CLA or a salt, ester or precursor thereof or milk fat enriched with c-9, t-11 CLA or a salt, ester or precursor thereof to a subject in need thereof. In one embodiment, the allergy is selected from contact dermatitis, eczema, hives (urticaria), allergic conjunctivitis, hay fever, allergic riinitis, airborne allergies including tree (e.g. birch pollen), weed (e.g. ragweed), and grass pollen allergies, latex allergies, food allergies (e.g. peanut, shellfish, milk protein), drug allergies (e.g. to penicillin), insect sting allergies (e.g. honeybee allergies, wasp allergies, hornet allergies, yellow jacket allergies, fire ant allergies), mold allergies (e.g. to alternaria, cladosporium, aspergillus, penicillium, helminthosporium, epicoccum, fusarium, mucor, rhizopus, and aureobasidium), dust mite allergies, animal allergies (e.g. household pets such as cats and dogs), allergic bronchopulmonary aspergillosis, occupational asthma, and episodic angioedema with eosinophilia.

Another aspect of the invention provides a method of treating or preventing eosinophilia comprising administering c-9, t-11 CLA or a salt, ester or precursor thereof or milk fat enriched with c-9, t-11 CLA or a salt, ester or precursor thereof to a subject in need thereof. In one embodiment, the eosinophilia is selected from airway, lung, blood and skin eosinophilia. In another embodiment, the eosinophilia is selected from eosinophilic ascites, eosinophilic cellulitis, eosinophilic fasciitis, eosinophilic gastroenteritis, coeliac disease, allergic colitis, eosinophilic esophagitis, eosinophilic pancreatitis, eosinophilic pneumonias, bronchiectasis, eosinophilic synovitis, nasal eosinophilia, tropical pulmonary eosinophilia, Churg Strauss syndrome, pulmonary eosinophilia, idiopathic hyper-eosinophilic syndrome, inflammatory bowel disease, eosinophilic cholangitis, eosinophilic leukaemia and other eosinophilic cancers, familial (hereditary eosinophilia), eosinophilic granuloma, sarcoidosis, eosinophilia-myalgia syndrome, cystic fibrosis, nasal polyposis, eosinophil meningitis, Wegener's granulomatosis, polyarteritis nodosa, rheumatoid arthritis, pemphigus vulgaris, bullous pemphigoid, dermatitis herpetiformis, erythema multiforme, eosinophilic cellulites, parasitic infections (Ascaris Toxocara canis, Filariasis, Anchylostomiasis, Trichinosis, Strongvloidiasis, Fascioliasis, Schistosomiasis).

Another aspect of the invention provides a method of treating or preventing a Th2 mediated condition comprising administering c-9, t-11 CLA or a salt, ester or precursor thereof or milk fat enriched with c-9, t-11 CLA or a salt, ester or precursor thereof to a subject in need thereof. In one embodiment, Th2 mediated conditions are selected from Th2 mediated asthma, allergies, eczema, microbial or parasite infection, and autoimmune diseases including ulcerative colitis.

Another aspect of the invention provides a method for treating or preventing a condition selected from those listed above with “steroid sparing” effect comprising the administration of c-9, t-11 CLA or a salt, ester or precursor thereof or milk fat enriched with c-9, t-11 CLA or a salt, ester or precursor thereof to a subject in need thereof. In one embodiment, the method allows the dose of any steroidal medication being administered to a subject to be reduced. In another embodiment, the invention provides a method for the treatment of a steroid-dependent condition such as corticosteroid dependent asthma, severe eczema or other eosinophilic disorders including eosinophilic gastroenteritis, eosinophilic pneumonia, and hyper-eosinophilic syndrome.

As used herein, the term “steroid sparing” is intended to mean that the dose of steroidal medication administered to a subject is able to be reduced to a level below that administered before the subject began taking a composition of the present invention. Preferably the dose is able to be reduced by at least 10, 20, 30, 40, 50, 60, 70, 80 or 90%.

The c-9, t-11 CLA and milk fat compositions useful herein may be formulated to allow for administration to a subject by any chosen route, including but not limited to oral, nasal, topical, subcutaneous, intramuscular, intravenous, or parenteral administration. Thus, a pharmaceutical composition of the invention may be formulated with appropriate pharmaceutically acceptable excipients, diluents or carriers selected with regard to the intended route of administration and standard pharmaceutical practice. For example, a composition of the invention can be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions can also be administered by inhalation (orally or intranasally), and are conveniently delivered in the form of a dry powder inhaler or an aerosol spray presentation from a pressurised container, pump, spray, atomiser or nebuliser, with or without the use of a suitable propellant as known in the art.

In one preferred embodiment, a composition for use according to the invention is formulated for ingestion, inhalation or topical application.

The compositions useful herein may be used alone or in combination with one or more other therapeutic agents. When used in combination with another therapeutic agent the administration of the two agents may be simultaneous or sequential. Simultaneous administration includes the administration of a single dosage form that comprises both agents and the administration of the two agents in separate dosage forms at substantially the same time. Sequential administration includes the administration of the two agents according to different schedules, preferably so that there is an overlap in the periods during which the two agents are provided. Suitable agents with which the compositions of the invention can be co-administered include bronchodilators e.g. beta-2 agonists, anticholinergic agents, or anti-inflammatory agents e.g. inhaled steroids, intranasal steroids, steroid creams and ointments, oral steroids and leukotriene antagonists and 5-lipoxygenase inhibitors, and other suitable agents known in the art.

In one embodiment of the invention, a pharmaceutical composition of the invention further comprises, or is formulated for administration (simultaneous or sequential) with, an agent selected from bronchodilators, corticosteroids, long-acting beta agonists, leukotriene modifiers and other suitable agents known in the art.

Additionally, it is contemplated that a composition in accordance with the invention may be formulated with additional active ingredients which may be of benefit to a subject in particular instances. For example, therapeutic agents that target the same or different facets of the disease process may be used.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject. However, by way of general example, the inventors contemplate administration of from about 1 mg to about 1000 mg per kg body weight of a milk fat composition of the invention is administered per day, preferably about 50 to about 100 mg per kg per day. In one embodiment, about 1 g to about 30 g of a milk fat composition of the invention is administered per day, preferably about 3 to about 7 g. It should be understood that a composition comprising c-9, t-11 CLA, rather than the CLA-enriched milk fat of the invention, may be administered in a lower daily dose than a CLA-enriched milk fat composition of the invention. For example, in one embodiment, the inventors contemplate administration of from about 0.05 mg to about 50 mg per kg body weight of a pharmaceutical composition of the invention comprising c-9, t-11 CLA.

It should be appreciated that administration may include a single daily dose or administration of a number of discrete divided doses as may be appropriate.

As used herein the term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition; for example reduction in leukocyte infiltration or eosinophilia, lesions, or preventing or otherwise reducing the risk of developing an allergic response, or disease symptom. The term “treat” also broadly includes the maintenance of good respiratory health for sensitive individuals and building stamina for disease prevention.

It should be understood that a person of ordinary skill in the art will be able without undue experimentation, having regard to that skill and this disclosure, to determine an effective amount of a composition of this invention for a given condition.

Various aspects of the invention will now be illustrated in non-limiting ways by reference to the following examples.

### EXAMPLES

### Mice

Eight to nine week old male and female C57BL/6 mice (University of Auckland, New Zealand) were kept on an ovalbumin (OVA)-free normal mouse chow diet from weaning up until they were assigned to a particular diet (control or experimental diet). Each diet group (n=6) contained an equal number of male and female mice. Throughout the study period mice were kept in an air-conditioned room with controlled humidity, temperature, and a 12 h light:dark cycle.

### Diets

CLA-enriched milk fat was prepared by supplementary free fatty acid feeding of pasture fed cows according to the method of Harfoot et al (32). The experimental diets were prepared using as a base the powdered AIN-93G formulation (33).

Healthy control mice were maintained on an unmodified AIN-93G diet and/or mouse chow. Soybean oil (which contains no CLA) was the dietary fat source in the AIN-93G diet. The final fat content of all treatment diets used in the Examples was maintained at the same level as the fat content of the control AIN-93G diet by reducing the soybean oil content of the treatment diet as required.

For Examples 1 to 3, two treatment diets were prepared where the AIN-93G diet was supplemented with 5% w/w of either normal milk fat or CLA-enriched milk fat, and the soybean oil content reduced such that the total fat content of the diets was unchanged.

For Examples 4 to 6, nine treatment diets were prepared where the AIN-93G diet was supplemented by 0.5%, 2% or 7% (w/w) with one of normal milk fat, CLA-enriched milk fat, or normal milk fat spiked with a synthetic CLA product derived from safflower oil (“syn-CLA”), and the soybean oil content reduced such that the total fat content of the diets was unchanged.

For Example 8, five treatment diets were prepared where the AIN-93G diet was supplemented with 0.07% (w/w) of synthetic c-9, t-11 CLA in free fatty acid or triglyceride form, synthetic t-10, c-12 CLA in free fatty acid or triglyceride form (Indofine Chemical Co., Hillsborough, N.J.), and the soybean oil content reduced such that the total fat content of the diets was unchanged.

The compositions of the normal milk fat, CLA-enriched milk fat and syn-CLA spiked milk fat used in the treatment diets are summarised in Tables 1 to 2. Data in Tables 1 to 2 was obtained using FAMES, extended FAMES, CLA and milk fat analyses known in the art.

TABLE 1
CLA content of treatment diets

| | | | CLA- | |
|---|---|---|---|---|
| | | Normal | enriched | syn-CLA spiked |
| Component | | (% w/w) | (% w/w) | (% w/w) |
| CLA-9,11 | c-9, t-11 CLA | 1.17 | 5.04 | 2.37 |
| CLA-A | | 0.1 | 0.4 | 0.09 |
| CLA-10,12 | t-10, c-12 CLA | — | — | 1.29 |
| CLA-B | | 0.09 | 0.21 | 0.11 |
| CLA-C + | c20:1 | 0.13 | 0.00 | 0.23 |
| CLA-C′ | | 0.00 | 0.15 | 0.00 |
| CLA-D | | 0.1 | 0.25 | 0.13 |
| CLA-E | | 0.13 | 0.29 | 0.25 |

| | | | |
|---|---|---|---|
| TOTAL CLA (all forms) | 1.59 | 6.34 | 4.24 |
| Ratio 9, 11 to Total | 73.58 | 79.50 | 55.90 |
| Ratio 10, 12 to Total | 0.00 | 0.00 | 30.42 |
| TOTAL CLA/milk fat | 1.59 | 6.34 | 4.24 |
| 9, 11 CLA/Milkfat | 1.17 | 5.04 | 2.37 |
| 10, 12 CLA/Milkfat | 0 | 0 | 1.29 |

Note:
CLA-A to CLA-E are c18:2 isomers of CLA and include cis-trans, trans-cis and trans-trans. An entry of “—” means the isomer was present in an amount below the quantitation limit.

TABLE 2
Extended fatty acids as determined by FAME analysis

| | Normal | CLA-enriched | syn-CLA spiked |
|---|---|---|---|
| Fatty Acid | (% w/w) | (% w/w) | (% w/w) |
| c4:0 | 3.2 | 3.2 | 3.48 |
| c6:0 | 2.3 | 1.7 | 2.16 |
| c8:0 | 1.3 | 1 | 1.22 |
| c10:0 | 2.8 | 2.2 | 2.66 |
| c10:1 | 0.3 | 0.2 | 0.29 |
| c12:0 | 3.2 | 2.5 | 3.07 |
| c12:1 | 0.2 | 0.1 | 0.07 |
| c13:0 br | 0.1 | 0 | 0.08 |
| c13:0 | 0.1 | 0.1 | 0.08 |
| c14:0 br | 0.2 | 0.1 | 0.14 |
| c14:0 | 10.9 | 9.1 | 10.53 |
| c14:1 | 0.9 | 0.8 | 0.9 |
| c15:0 iso br | 0.4 | 0.3 | 0.36 |
| c15:0 ante-iso br | 0.6 | 0.6 | 0.54 |
| c15:0 | 1.3 | 1.2 | 1.28 |
| c16:0 br | 0.2 | 0.2 | 0.22 |
| c16:0 | 30.6 | 19.7 | 29.97 |
| c16:1 | 1.8 | 3 | 1.68 |
| c17:0 iso br | 0.6 | 0.6 | 0.51 |
| c17:0 ante-iso br | 0.4 | 0.5 | 0.4 |
| c17:0 | 0.8 | 0.5 | 0.89 |
| c17:1 | 0.3 | 0.3 | 0.27 |
| c18:0 | 10.5 | 4.6 | 10.25 |
| c18:1 n-9 | 16.6 | 11.9 | 16.5 |
| c18:1 n-7 | 4.7 | 22.9 | 4.68 |
| c18:2 n-6 | 1.4 | 2.1 | 1.29 |
| c18:3 n-3 | 0.8 | 0.4 | 0.73 |
| c18:2 conj (CLA) | 1.2 | 5.3 | 2.56 |
| c18:4 + CLA isomers | 0 | 0 | 1.41 |
| c20:0 | 0.2 | 0.1 | 0.04 |
| c20:1 n-11 | 0.1 | 0.1 | 0.14 |
| c20:1 n-9 | 0 | 0.1 | 0 |
| c20:2 n-6 | 0 | 0 | 0 |
| c20:3 n-3 | 0.1 | 0 | 0.05 |
| c20:4 n-6 (AA) | 0.1 | 0 | 0.06 |
| c20:3 n-3 | 0 | 0.1 | 0 |
| c20:4 n-3 | 0.1 | 0.2 | 0.04 |
| c20:5 n-3 (EPA) | 0.1 | 0.2 | 0.08 |
| c22:0 | 0.1 | 0.1 | 0 |
| c22:1 n-13, n-11 | 0 | 0.1 | 0 |
| c22:2 n-9 | 0 | 0 | 0 |
| c22:4 n-6 | 0 | 0 | 0 |
| c22:5 n-3 | 0.1 | 0 | 0.09 |
| c24:0 | 0 | 0 | 0 |
| c22:6 n-3 (DHA) | 0 | 0 | 0 |
| c24:1 | 0 | 0 | 0 |

Note:
c18:1 n-7 provides an estimate of the c18:1 trans fatty acid content

Fresh diet was provided biweekly, and mice had free access to food and water throughout the study. The body weights of both female and male mice remained relatively constant irrespective of which diet they were fed, differing by no more than 22%. Any differences in net weight gain were not statistically significant, except the body weights of males fed the lowest dose of CLA-enriched milk fat were slightly increased (P<0.05) compared to those of males fed the highest levels of CLA-enriched milk fat and syn-CLA-spiked milk fat. Male mice were generally 11 to 50% heavier than female mice.

### Sensitization and Allergen Exposure Protocol

Body weights were determined, and blood samples collected via the tail vein, prior to assignment of mice to particular diets. After two weeks on an assigned diet mice were immunized with two intraperitoneal (i.p.) injections of 20 μg of OVA (chicken egg albumin grade V; Sigma Chemical Co., St Louis, Mo.) complexed with 2 mg of Imject Alum (Al(OH)₃/Mg(OH)₂; Pierce Rockford Ill.) in a total volume of 100 μl of PBS on days 0 and 14. Two weeks after the 2nd injection mice were anaesthetized by i.p. injection of a mixture of ketamine and xylazine (Phoenix, Auckland, New Zealand), and challenged intranasally with 100 μg of OVA in 50 μl of PBS. Undiseased control mice were immunized and challenged with PBS using a similar regime. Body weights were determined, and blood, bronchoalveolar lavage (BAL) fluid, and lung tissue samples were collected 6 days after the intranasal challenge. Blood was collected by cardiac puncture after deeply anaesthetizing mice by i.p. injection of a mixture of ketamine and xylazine. Serum was separated from blood samples, and stored at −80° C.

Assessment of Inflammatory Cell Infiltration into the Lung

Bronchoalveolar lavage was performed immediately after euthanasia by flushing 1 ml of PBS containing 1% heat inactivated fetal calf serum (lavage buffer) thrice through the lung and airways of mice via the cannulated trachea. The recovered BAL was pooled for each animal, centrifuged at 1,500 rpm at 4° C., and the supernatant stored at −80° C. The cell pellets were resuspended in 1 ml of lavage buffer, and total cell numbers were counted using a hemocytometer. BAL cells were centrifuged onto poly-L-lysine-coated glass slides using a cytospin, and stained with Diff-Quik stain (Dade Behring Inc. USA). Differential cellular counts were made by counting ≧300 cells under light microscopy (Nikon E200 microscope), using standard morphological criteria.

### Histochemistry

Following BAL, the right lobes of lungs were immediately frozen in dry ice and stored at −80° C. for protein and Western blot analysis. The left lobes of lungs were fixed in 4% paraformaldehyde in 0.1 M PBS (pH 7.4) overnight and embedded either in optimum cutting temperature compound (OCT, Tissue Tek) and kept frozen at −80° C. until cryosectioning for immunohistochemistry, or in paraffin for routine histopathological analysis. Some were stained with a combined hematoxylin-Biebrich Scarlet solution by Luna's method for eosinophils to detect eosinophil granules (34) or with Alcian Blue-Periodic Acid Schiff for the detection of acid and neutral mucins and identification of goblet cells.

### Measurement of OVA-Specific Immunoglobulins

OVA-specific IgG1 in serum samples was measured by standard ELISA employing 96 well microtitre plates (Nunc Maxisorp). Plates were coated with 100 μl of 0.1 M carbonate buffer, pH 9.5, containing 10 μg/ml of OVA (Sigma) overnight at 4° C. After 3 washes with PBS-T (PBS with 0.05% Tween-20) plates were blocked with 200 μl of 3% BSA in PBS, pH 7.2, at room temperature for 90 min. One hundred microlitres of each serum sample (diluted 1:40) was added in triplicate to the wells, and incubated for 2 h at 37° C. Wells were washed four times with PBS-T and 100 μl of goat anti-mouse IgG1-HRP conjugated antibody (Bethyl Laboratories, USA) was added at 1:50,000 dilution. Plates were incubated for 1 h at 37° C., washed 5 times with PBS-T, and 100 μl of peroxidase enzyme substrate o-phenylene diamine (OPD, Sigma Chemical Co, St Louis, Mo.) was added to each well. The colour reaction was stopped after ˜20 min by addition of 50 μl of 4 M H₂SO₄ to each well. Absorbance was read at 490 nm in a BioRad microplate ELISA reader. Positive and negative sera were used as controls.

OVA-specific IgE was measured as for OVA-specific IgG1 except serum samples were diluted 1:5, and OVA was coated on plates at 100 μg/ml (35). A biotin-conjugated rat anti-mouse IgE monoclonal antibody (diluted 1:200; Pharmingen, San Diego, Calif.) was used to detect OVA-specific IgE antibody, and was detected with avidin-HRP conjugate (diluted 1:250) followed by development with OPD.

### Statistical Analysis

Data are expressed as the mean+SEM, and statistical significance was determined by the Student's t test. A value of P<0.05 was taken as significant.

### Results

### Example 1

### Feeding of a Milk Fat Diet Enriched in c-9, t-11 CLA Diminishes Leukocyte Infiltration into the Lungs of Allergen-Challenged Mice

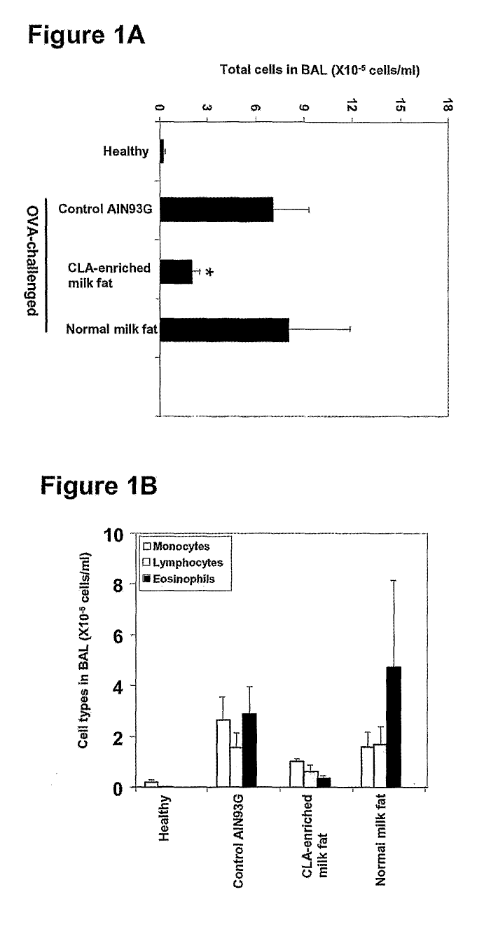

Mice were fed one of three diets, namely a control AIN93G diet, a CLA-enriched milk fat diet containing 5.04% of the milk fat fatty acids as c-9, t-11 CLA and a normal milk fat diet containing 1.17% of the milk fat fatty acids as c-9, t-11 CLA. After two weeks on each assigned diet, mice were immunized by i.p. injection with 20 μg of OVA, followed two weeks later by a further OVA injection. Two weeks after the 2nd injection mice were challenged intranasally with 100 μg of OVA, and leukocytes that had infiltrated the lung were collected by BAL six days later. The allergen challenge led to a massive increase in the leukocyte content of the lungs of mice fed the control AIN93G diet, and the normal milk fat diet (FIG. 1A). The CLA-enriched milk fat diet had a suppressive effect on allergen-induced accumulation of leukocytes into the lung. Total BAL cell counts were reduced by 72 (P<0.01) and 75% (P<0.05), respectively, compared to those obtained from mice fed the control AIN93G diet, and the normal milk fat diet (FIG. 1A). The CLA-enriched milk fat diet suppressed the accumulation of eosinophils by 88 (P<0.01) and 93% (P<0.05), respectively, compared to the numbers of eosinophils in the BAL of mice fed the control ATN93G diet, and the normal milk fat diet (FIG. 1B). The decrease in eosinophils was accompanied by a marked 61 (P<0.01) and 35% (P>0.05) reduction in the numbers of monocytes/macrophages, and a similar 61 (P<0.05) and 64% (P<0.05) reduction in the numbers of lymphocytes, compared to the numbers of monocytes/macrophages and lymphocytes in the BAL of mice fed the control AIN93G diet, and the normal milk fat diet, respectively (FIG. 1B). The BAL from healthy PBS-treated control mice had a very low cellular content (FIG. 1A) comprised almost entirely of monocytes/macrophages (FIG. 1B). Neutrophil numbers in the BAL were very low irrespective of the type of diet, and did not increase significantly following allergen challenge, and hence were not further analysed.

### Example 2

### CLA-Enriched Milk Fat Induces Cytolysis of BAL Eosinophils, and Clearance of Eosinophil Debris by Monocytes/Macrophages

Eosinophil cytolysis and degranulation are characteristic features of asthma in humans, and are believed to play a role in causing tissue damage due to the release of cytotoxic granule contents (36). However, eosinophils have not been convincingly demonstrated to undergo cytolysis or degranulation in the common mouse models of asthma. In accord, the eosinophils in the BAL of OVA-challenged mice fed the control AIN93G diet were perfectly normal in appearance. In contrast, those of OVA-challenged mice fed the CLA-enriched milk fat diet had often undergone cytolysis, as evidenced by chromatolysis, loss of plasma membrane integrity, and release of membrane-bound specific granules that were visualized as clusters of free eosinophil granules (cfegs). Cfegs were often seen to have been phagocytosed by monocyte/macrophages, with some macrophages containing up to six cfegs. Some macrophages were heavily vacuolated suggesting they may have engulfed eosinophil plasma membrane fragments. In contrast, eosinophils in the BAL of OVA-challenged mice fed the normal milk fat diet had a normal morphology.

### Example 3

### CLA-Enriched Milk Fat Diminishes Allergen-Specific Ig Responses

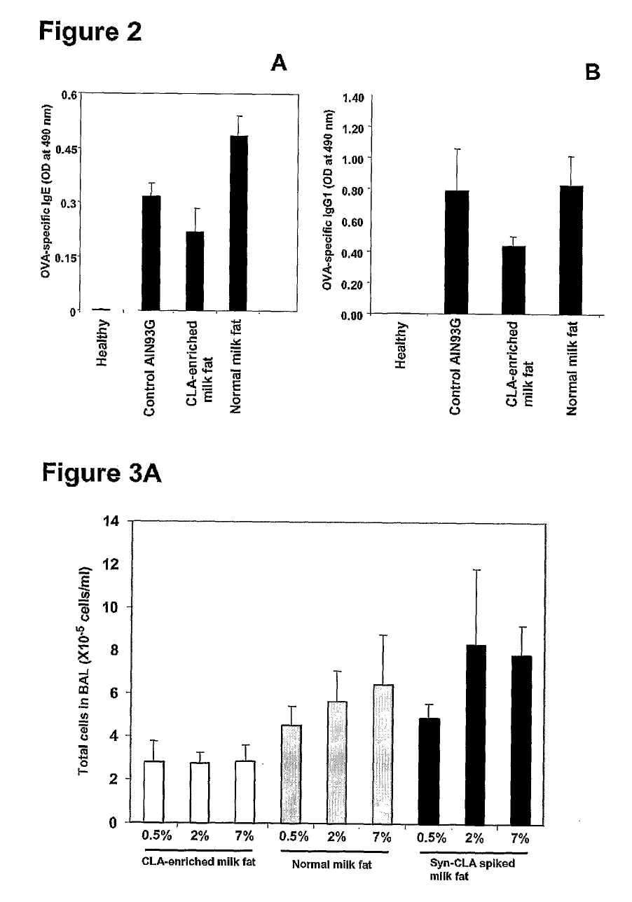

Challenge with allergen led to a massive increase (P<0.001) in the levels of OVA-specific IgE (FIG. 2A) and OVA-specific IgG1 (FIG. 2B) in the sera of mice fed the control AIN93G diet, and the normal milk fat diet. The CLA-enriched milk fat diet suppressed the increase in OVA-specific IgE by 30 (P<0.05) and 55% (P<0.001), and OVA-specific IgG1 by 45 (P<0.05) and 48% (P<0.01), respectively, compared to levels in the sera of mice fed the control AIN93G diet, and the normal milk fat diet.

### Example 4

### CLA-Enriched Milk Fat Blocks Leukocyte Infiltration at Low Doses, and is Superior to a Synthetic Seed Form of CLA (“syn-CLA”)

Two previous publications reported that feeding of an approximately equal mixture of synthetic cis-9, trans-11 and trans-10, cis-12 isomers of synthetic seed CLA (0.25 g CLA/100 g of diet) for one to two weeks prior to and during OVA sensitization reduced allergen-induced histamine and PGE2 release from allergen-sensitized guinea pig tracheae (23,24), but did not affect allergen-induced tracheal contractions (23). The lack of an effect on tracheal contraction and the decrease in PGE2, which is an inhibitor of the early and late bronchoconstrictor response to inhaled allergen (37), suggests that synthetic seed CLA has the potential to exacerbate the symptoms of asthma. To test the latter possibility, and to compare the effectiveness of different doses of CLA-enriched milk fat with synthetic seed-derived CLA, mice were fed diets composed of CLA-enriched milk fat, normal milk fat, and normal milk fat spiked with syn-CLA, where the milk fat content of each diet ranged from 0.5, 2, and 7%. The CLA-enriched milk fat used in the study was composed of 6.34% CLA (w/w), thus each of the latter three CLA-enriched milk fat diets contained 0.032, 0.13, and 0.44% CLA (w/w). The normal milk fat used in the study was composed of 1.59% CLA (w/w), thus each of the latter three normal milk fat diets contained 0.008, 0.032, and 0.113% CLA (w/w). The syn-CLA-spiked normal milk fat used in the study was composed of 1.59% milk CLA and 2.65% syn-CLA to give a total of 4.24% CLA (w/w), thus each of the latter three syn-CLA-spiked normal milk fat diets contained 0.008, 0.032, and 0.113% milk CLA, and 0.0132, 0.052 and 0.182% syn-CLA, or 0.021, 0.081 and 0.294% CLA in total (w/w). The OVA sensitization and challenge, and feeding regime, were as described above.

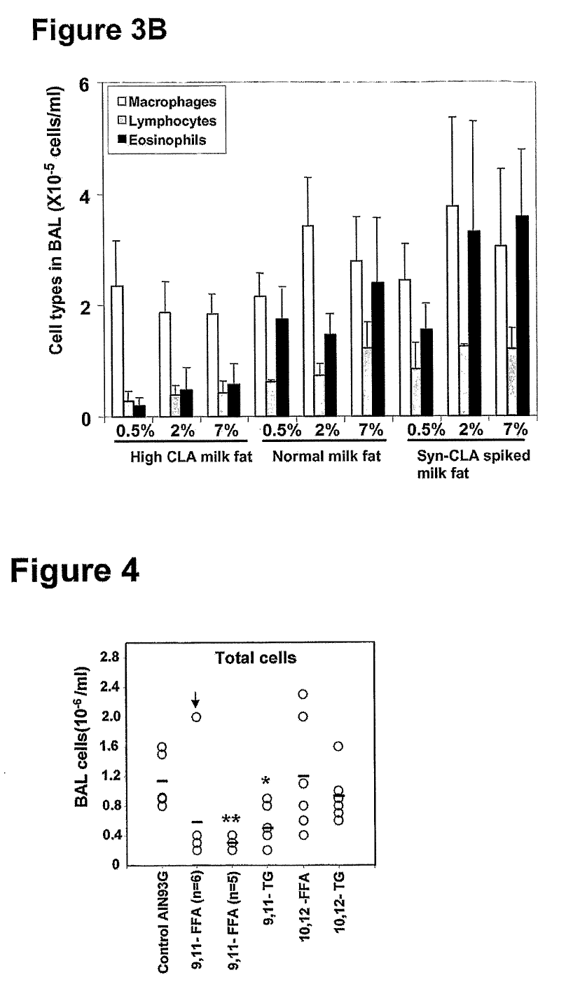

Allergen challenge led to large numbers of leukocytes infiltrating the lungs of mice fed the normal milk fat control diet (FIG. 3A). The cellular content of the BAL increased with increasing doses of milk fat in the diet. Thus, there was a 43% increase in the numbers of leukocytes in the BAL of mice fed a 7% milk fat diet versus those fed a 0.5% milk fat diet. Once again, the CLA-enriched milk fat diet had a suppressive effect on allergen-induced accumulation of leukocytes into the lung. Total BAL cell counts for mice fed the lowest and highest dose of CLA-enriched milk fat diet were reduced by 38 (P<0.05) and 56% (P<0.05), respectively, compared to those obtained from mice fed the lowest and highest dose of normal milk fat diet, respectively (FIG. 3A). The cellular content of the BAL did not increase with increasing doses of CLA-enriched milk fat. In contrast, the lowest dose of syn-CLA-spiked milk fat had no apparent therapeutic benefit as the cellular content of the BAL was similar to that obtained by feeding normal milk fat. Increased doses of syn-CLA-spiked milk fat increased the cellular content of the BAL by 70% (P<0.01) compared to low doses of syn-CLA-spiked milk fat, and by 30% (P>0.05) compared to the highest does of normal milk fat.

The cellular content of the BAL of mice fed the CLA-enriched milk fat diet was greater than that of undiseased control mice, hence the leukocytes in the BAL were phenotyped to determine the relative numbers of leukocyte subsets, in particular the numbers of potentially pathogenic eosinophils (FIG. 3B). The BAL of mice fed the normal milk fat and syn-CLA-spiked milk fat diets contained high numbers of monocytes/macrophages and eosinophils in almost equal proportions, and lesser but nevertheless high numbers of lymphocytes. The BAL of mice fed high doses of syn-CLA-spiked milk fat were found to contain the highest numbers of eosinophils, in accord with the high cellular content of the BAL. In marked contrast to the latter two diets, CLA-enriched milk fat skewed the leukocyte profile in favour of monocytes/macrophages that are almost the sole residents of the lungs of healthy mice. Thus, whereas the macrophage content of the BAL of mice fed low doses CLA-enriched milk fat diet was similar to that of mice fed a corresponding amount of the other two diets, the eosinophil and lymphocyte content was reduced by 87 to 90 (P<0.01 to 0.001), and 56 to 68% (P<0.01 to 0.05), respectively. The eosinophil and lymphocyte content was reduced by 76 to 84% (P<0.01 to 0.05) and 64 to 65% (P<0.01), respectively, when a comparison was made of the effects of the highest doses of each diet.

### Example 5

### CLA-Enriched Milk Fat Inhibits Airway Changes Including Leukocyte Infiltration, Bronchoconstriction, Airway Epithelial Cell Hypertrophy, Goblet Cell Metaplasia and Mucous Secretion

As in humans, the airways of OVA-sensitized mice undergo major pathologic changes following challenge with allergen (36). Such changes were epitomized in asthmatic animals fed the normal milk fat and syn-CLA-spiked milk fat diets. Thus, in addition to massive peribronchial and perivascular infiltrates of leukocytes, there was marked epithelial cell hypertrophy, and goblet cell metaplasia. Furthermore, the alcian blue-periodic acid Schiff double staining method showed that the airway epithelial content of neutral mucopolysaccharides stained “red” with Schiffs reagent increased dramatically in response to allergen challenge. In marked contrast, similar changes to the lungs of allergen challenged mice fed the CLA-enriched milk fat diet were minimal. Only traces of neutral mucopolysaccharides were evident.

The level of Schiff reagent staining of the epithelium in the airways was semi-quantitatively determined and recorded as the mucus index score (Table 3). All three precentages of CLA-enriched milk fat significantly inhibited (51 to 66%) mucus production. The syn-CLA-spiked milk fat diets had no significant effect.

TABLE 3
Mucus index scores

| | | P value |
|---|---|---|
| | Mucus index score | (Compared to |
| Diet group | (Mean ± SEM) | other Diet Group) |
| Control AIN-93 G | 3.54 ± 0.23 | |
| A1 (0.5% CLA enriched | 1.74 ± 1.36 | P < 0.05 (A6, A9, |
| milk fat) | | Control AIN-93G) |
| A2 (2% CLA enriched milk | 1.2 ± 1.01 | P < 0.05 (A8) |
| fat) | | P < 0.01 (A9, |
| | | Control AIN-93G) |
| | | P < 0.001 (A6) |
| A3 (7% CLA enriched milk | 1.52 ± 1.43 | P < 0.05 (A6, A9, |
| fat) | | Control AIN-93G)) |
| A4 (0.5% synthetic-CLA- | 2.53 ± 1.47 | NS |
| spiked milk fat) | | |
| A5 (2% synthetic-CLA- | 3.08 ± 1.10 | NS |
| spiked milk fat) | | |
| A6 (7% synthetic-CLA- | 3.66 ± 0.372 | NS |
| spiked milk fat) | | |

The level of Schiff staining of airway epithelia was semi-quantitatively determined to give a mucus index score as described (38), but modified in that Schiff staining was scored by microscopic viewing of airways. A minimum of 4 to 6 representative transversely or sagittally sectioned airways were graded per animal using a scale of 0 (no staining), 1 (25% or less of the airway epithelium stained), 2 (26-50% of the airway epithelium stained), 3 (51-75% of the airway epithelium stained), and 4 (75% of the airway epithelium stained). Airways were analysed only when the complete circumference of the airway could be visualized, and those that opened directly into an alveolar space were not included.

The bronchial airways of mice fed the CLA-enriched milk fat diet appeared to be less constricted than those of mice fed the other two diets. The airways of mice fed the CLA-enriched milk fat diet were more similar to those of undiseased mice. There were considerably less eosinophils in the lungs, and those present showed signs of cytolysis. Macrophages could be detected that had engulfed large numbers of cfegs in common with the situation in the BAL. In contrast, blood smears established that eosinophils at peripheral sites had a normal morphology. Changes to the lung were scored for inflammation, perivascular/peribronchiolar infiltrates, beneficial presence of phagocytic macrophages, airway epithelial hypertrophy, goblet-cell hyperplasia, constriction of bronchioles, and mucus hypersecretion to give an overall histopathology score (Table 4). The above results indicate that CLA-enriched milk fat is able to inhibit both allergen-specific remodelling and inflammation of the lung.

TABLE 4
Histopathology scores of lung airway changes

| | | P value |
|---|---|---|
| | Histopathology score | (Compared to |
| Diet Group | (Mean ± SEM) | other Diet Groups) |
| Healthy mice (not OVA | 0.05 ± 0.04 | — |
| challenged) | | |
| Control AIN-93G | 3.82 ± 0.18 | — |
| (OVA challenged) | | |
| A1 (0.5% CLA-enriched | 1.36 ± 0.76 | P > 0.05 (A2, A3) |
| milk fat) | | P < 0.05 (A4) |
| | | P < 0.01 (A5-A7) |
| | | P < 0.001 (A8, A9, |
| | | Control AIN-93G) |
| A2 (2% CLA-enriched | 1.85 ± 0.52 | P > 0.05 (A1, A3) |
| milk fat) | | P < 0.05 (A4) |
| | | P < 0.01 (A5) |
| | | P < 0.001 (A6-A9, |
| | | Control AIN-93G) |
| A3 (7% CLA-enriched | 1.68 ± 0.72 | P > 0.05 (A1, A2) |
| milk fat) | | P < 0.05 (A4) |
| | | P < 0.01 (A5-A8) |
| | | P < 0.001 (A9, |
| | | Control AIN-93G) |
| A4 (0.5% normal milk | 3.25 ± 0.73 | NS (not |
| fat) | | significantly less) |
| A5 (2% normal milk fat) | 3.35 ± 0.40 | NS |
| A6 (7% normal milk fat) | 3.87 ± 0.15 | NS |
| A7 (0.5% syn-CLA- | 3.62 ± 0.22 | NS |
| spiked milk fat) | | |
| A8 (2% syn-CLA- | 3.72 ± 0.46 | NS |
| spiked milk fat) | | |
| A9 (7% syn-CLA- | 3.78 ± 0.16 | NS |
| spiked milk fat) | | |

The histopathology score was determined from inspection of alcian blue-PAS stained paraffin embedded sections of the left lung of each animal. Lung inflammation, perivascular/peribronchiolar infiltrates, airway epithelial hypertrophy, goblet-cell hyperplasia, constriction of bronchioles, mucin hypersecretion, and beneficial presence of phagocytic macrophages were graded on a scale of 0 (no change) to 4 (marked change). Each animal received an overall histopathology score based on summation of individual scores for each criteria. All slides were scored in a blinded fashion (blinded to diet treatment/group assignment), and scores were presented as the mean±SEM of 4-6 animals/group.

### Example 6

### CLA-Enriched Milk Fat Displays No Apparent Organ Toxicity, Whereas High Levels of Syn-Synthetic Seed CLA-Spiked Milk Fat Induces Fatty Liver Disease

Visual analysis of the spleens and livers of mice fed high doses of milk fats indicated there were no obvious signs of toxicity, except in the case of mice fed high doses of syn-CLA-spiked milk fat. While the spleens and livers of all mice appeared similar with similar organ weights per body weight, there was one exception. The livers of mice fed the highest level of syn-CLA-spiked milk fat were on average 58% (P<0.001) heavier than those of mice fed the highest levels of normal milk fat or CLA-enriched milk fat. They were very pale in colour suggesting elevated deposition of fat. Histological analysis of the livers of mice fed syn-CLA-spiked milk fat revealed panlobular macrovesicular steatosis (fat accumulation). There was massive vacuolization due to fat deposition, but only mild hepatic inflammation. Numerous hepatocytes contained granular to amorphous material reminiscent of Mallory bodies that are associated with liver steatosis (39). In contrast, the livers of mice fed the other two milk-based diets had normal histology.

### Example 7

### A Broad Dose Range of CLA-Enriched Milk Fat Diminishes Allergen-Specific Ig Responses Compared to Milk Fat and Syn-CLA-Spiked Milk Fat

Different doses of CLA-enriched milk fat were examined for their ability to diminish allergen-specific Ig responses compared to similar doses of milk fat and syn-CLA-spiked milk fat. CLA-enriched milk fat diets at 0.5, 2, and 7% suppressed the increase in OVA-specific IgE by 60, 50, and 54.8% (Table 5), and OVA-specific IgG1 by 31, 31, and 38% (Table 6), respectively, compared to levels in the sera of mice fed the normal milk fat diet at 0.5, 2, and 7%, respectively, and by 62, 51, and 47% (IgE, Table 5), and 27, 41, and 29% (IgG1, Table 6), respectively, compared to levels in the sera of mice fed the syn-CLA-spiked milk fat diets at 0.5, 2, and 7%.

TABLE 5
Allergen-specific IgE responses in OVA challenged
mice fed experimental diet

| | OVA-specific IgE | P value (Compared to |
|---|---|---|
| Diet Group | (Mean ± SEM) | other Diet Groups) |
| A1 (0.5% CLA enriched | 0.47 ± 0.11 | P < 0.05 (A4& A8) |
| milk fat) | | P < 0.001 (A5, A6, |
| | | A7& A9) |
| A2 (2% CLA enriched milk | 0.55 ± 0.09 | P < 0.01 (A5) |
| fat) | | P < 0.001 (A6, |
| | | A7 & A9) |
| A3 (7% CLA enriched milk | 0.59 ± 0.11 | P < 0.01 (A5) |
| fat) | | P < 0.001 (A6, |
| | | A7 & A9) |
| A4 (0.5% normal milk fat) | 1.17 ± 0.35 | Refer above |
| A5 (2% normal milk fat) | 1.10 ± 0.12 | Refer above |
| A6 (7% normal milk fat) | 1.31 ± 0.23 | Refer above |
| A7 (0.5% syn-CLA-spiked | 1.25 ± 0.16 | Refer above |
| milk fat) | | |
| A8 (2% syn-CLA-spiked | 1.12 ± 0.50 | Refer above |
| milk fat) | | |
| A9 (7% syn-CLA-spiked | 1.12 ± 0.10 | Refer above |
| milk fat) | | |

TABLE 6
Allergen-specific IgG responses in OVA challenged mice fed
experimental diets

| | OVA-specific | |
|---|---|---|
| | IgG1 | P value (Compared to |
| Diet Group | (Mean ± SEM) | other Diet Groups) |
| A1 (0.5% CLA enriched | 1.62 ± 0.20 | P < 0.01 (A5 & A6) |
| milk fat) | | P < 0.001 (A4, A7-A9) |
| A2 (2% CLA enriched milk | 1.63 ± 0.48 | P < 0.05 (A4, A5, |
| fat) | | A7& A9) |
| | | P < 0.01 (A6 & A8) |
| A3 (7% CLA enriched milk | 1.73 ± 0.26 | P < 0.05 (A7) |
| fat) | | P < 0.01 (A4-A6 & A9) |
| | | P < 0.001 (A8) |
| A4 (0.5% normal milk fat) | 2.36 ± 0.10 | Refer above for A1-A3 |
| | | P < 0.05 (A8) |
| A5 (2% normal milk fat) | 2.36 ± 0.19 | Refer above for A1-A3 |
| | | P < 0.05 (A8) |
| A6 (7% normal milk fat) | 2.78 ± 0.60 | Refer above |
| A7 (0.5% syn-CLA-spiked | 2.21 ± 0.14 | Refer above for A1-A3 |
| milk fat) | | P < 0.01 (A8) |
| A8 (2% syn-CLA-spiked | 2.78 ± 0.22 | Refer above |
| milk fat) | | |
| A9 (7% syn-CLA-spiked | 2.43 ± 0.24 | Refer above |
| milk fat) | | |

### Example 8

### Synthetic c-9, t-11 CLA Isomer Reduces Lung Inflammation Mice, Whereas the t-10, c-12 CLA Isomer is Ineffective

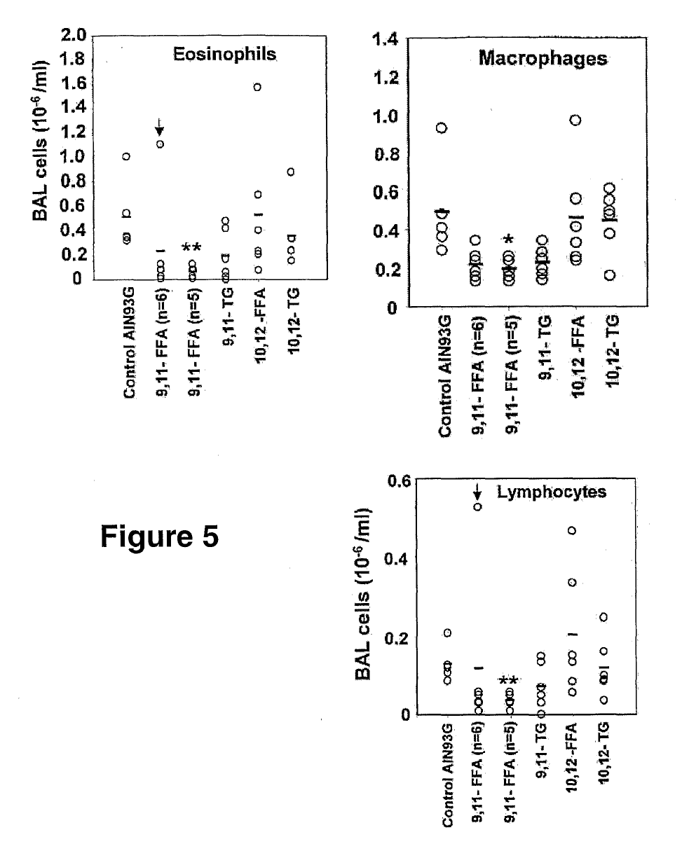

Mice were fed diets containing 0.07% of each of the c-9, t-11 and t-10, c-12 CLA isomers in both the free fatty acid and triglyceride forms. The diet supplemented with the c-9, t-11 isomer in either its free fatty acid or triglyceride forms suppressed allergen-induced accumulation of leukocytes into the lung. Total BAL cell counts were on average reduced by 74 (P<0.01) and 56% (P<0.05), respectively, compared to those obtained from mice fed the control diet (FIG. 4). In contrast, there was no significant difference in the total BAL count of mice fed the t-10, c-12 CLA isomer compared to the control diet. The free fatty acid and triglyceride forms of the c-9, t-11 isomer on average suppressed the accumulation of eosinophils by 87 (P<0.01) and 62% (P>0.05), respectively, compared to the control diet, however significance was only reached in the former comparison (FIG. 5). In contrast, there was no significant difference in the eosinophil count of mice fed the t-10, c-12 CLA isomer compared to the control diet. The decrease in eosinophils was accompanied by a 61 (P<0.05) and 53% (P>0.05) reduction in the numbers of monocytes/macrophages compared to the numbers of monocytes/macrophages in the BAL of mice fed the control diet. There was a similar 72 (P<0.01) and 43% (P>0.05) reduction in the numbers of lymphocytes, compared to mice fed the control diet. The latter comparisons only reached significance for the free fatty acid form of the c-9, t-11 isomer. In contrast, there was no significant difference in the monocyte/macrophages and lymphocyte counts of mice fed the t-10, c-12 CLA isomer compared to the control diet. Thus, the c-9, t-11 CLA isomer, in particular the free fatty acid form, suppresses lung inflammation in response to allergen.

### Example 9

### Synthetic Free Fatty Acid c-9, t-11 CLA Isomer Reduces Mucus Hypersecretion

The bronchiole airways of the lungs of mice in example 7 were examined for mucus production (Table 7). The free fatty acid form of the c-9, t-11 isomer on average suppressed mucus production by 32% compared to the control diet, however significance was not reached (Table 4), whereas the triglyceride form displayed negligible inhibition (15%). The free fatty acid and triglyceride forms of the t-10, c-12 CLA isomer also displayed negligible inhibition (7 and 15%, respectively).

TABLE 7
Mucus index scores

| | Mucus Index | P value |
|---|---|---|
| | Score | (Compared to |
| Diet group | (Mean ± SEM) | control AIN-93G diet) |
| Synthetic c-9, t-11 CLA isomer | 2.04 ± 1.13 | NS (not significant) |
| Free fatty acid | | |
| Synthetic t-10, c-12 CLA isomer | 2.80 ± 1.35 | NS |
| Free Fatty Acid | | |
| Synthetic c-9, t-11 CLA isomer | 2.58 ± 0.94 | NS |
| Triglyceride | | |
| Synthetic t-10, c-12 CLA isomer | 2.57 ± 1.16 | NS |
| Triglyceride | | |
| Control AIN-93G | 3.02 ± 1.05 | NS |

### Example 10

### Synthetic Free Fatty Acid c-9, t-11 CLA Isomer Reduces Overall Lung Pathology

Changes to the lung were scored for inflammation, perivascular/peribronchiolar infiltrates, beneficial presence of phagocytic macrophages, airway epithelial hypertrophy, goblet-cell hyperplasia, constriction of bronchioles, and mucus hypersecretion to give an overall histopathology score (Table 8). The results below indicate that the free fatty acid form of the c-9, t-11 isomer is able to significantly inhibit both allergen-specific remodelling and inflammation of the lung, whereas the t-10, c-12 CLA isomer was not effective. The histopathology score of 2.02 for the free fatty acid form of the c-9, t-11 isomer is higher, but not significantly different from that recorded for 0.5% CLA-enriched milk fat (1.36), and 2% CLA-enriched milk fat (1.85), respectively. The c-9, t-11 isomer inhibited inflammation to a similar extent as CLA-enriched milk fat, but had less of an effect on allergen-specific remodelling.

TABLE 8
Histopathology scores of lung airway changes

| | Histopathology | P value |
|---|---|---|
| | score | (Compared to |
| Diet group | (Mean ± SEM) | control AIN-93G diet) |
| Synthetic c-9, t-11 CLA isomer | 2.02 ± 0.66 | P < 0.01 |
| Free fatty acid | | [P < 0.05 (all other |
| | | diet groups)] |
| Synthetic t-10, c-12 CLA | 3.28 ± 0.46 | NS |
| isomer Free Fatty Acid | | |
| Synthetic c-9, t-11 CLA isomer | 3.08 ± 0.69 | NS |
| Triglyceride | | |
| Synthetic t-10, c-12 CLA | 3.20 ± 0.79 | NS |
| isomer | | |
| Triglyceride | | |
| Control AIN-93G | 3.70 ± 0.19 | NS |

### Example 11

### CLA Isomers in Either Free Fatty Acid or Triglyceride Forms have No Significant Effect on Allergen-Specific Ig Responses

Diets supplemented with the c-9, t-11 and t-10, c-12 CLA isomers in either their free fatty acid or triglyceride forms had no significant effect on the increase in OVA-specific IgE (Table 9) and IgG1 (Table 10) compared to mice fed the control AIN-93 diet.

TABLE 9
Allergen-specific IgE responses in OVA challenged mice

| | Control | | | | 10,12- |
|---|---|---|---|---|---|
| IgE Values* | AIN 93G | 9,11-FFA | 9,11-TG | 10,12-FFA | TG |
| Average | 1.25 | 0.89 | 1.17 | 1.25 | 1.29 |
| SEM | 0.37 | 0.15 | 0.32 | 0.34 | 0.28 |

*All IgE values for CLA isomer-fed mice are non-significant when compared with the control diet group.
FFA, free fatty acid;
TG, triglyceride.

TABLE 10
Allergen-specific IgG responses in OVA challenged mice

| | Control | | | | 10,12- |
|---|---|---|---|---|---|
| IgG Values* | AIN 93G | 9,11-FFA | 9,11-TG | 10,12-FFA | TG |
| Average | 2.14 | 1.91 | 1.96 | 1.95 | 1.98 |
| SEM | 0.22 | 0.27 | 0.15 | 0.37 | 0.19 |

*All IgG1 values for CLA isomer-fed mice are non-significant when compared with the control diet group.
FFA, free fatty acid;
TG, triglyceride.

For comparison, the allergen-specific IgE and IgG values for healthy control mice injected with PBS was 0.24±0.03 and 0.02±0.04, respectively.

### INDUSTRIAL APPLICATION

The present invention has utility in treating or preventing conditions associated with one or more of leukocyte infiltration, eosinophilia, IgE secretion, airway remodelling, bronchoconstriction and mucus hypersecretion. The described compositions may be employed as food or drink additives, nutritional products, dietary supplements, neutraceuticals and pharmaceuticals. The described compositions and methods of the invention may be employed to treat or prevent one or more of the conditions discussed above.

Those persons skilled in the art will understand that the above description is provided by way of illustration only and that the invention is not limited thereto.

### REFERENCES

1. Kay A B. Allergy and allergic diseases. N. Eng. J. Med. 344: 30-37, 2001.
2. Christodoulopoulos P, Cameron L, Durham S, Hamid, Q. Molecular pathology of allergic disease. II. Upper airway disease. J. Allergy Clin. Immunol. 105: 211-223, 2000.
3. Lamblin C, Gosset P, Salez, F, Vandezande L M, Perez T, Darras J, Janin A, Tonnel A B, Wallaert B. Eosinophilic airway inflammation in nasal polyposis. J. Allergy Clin. Immunol. 104: 85-92, 1999.
4. Erjefalt J S, Greiff L, Andersson M, Matsson E, Petersen H, Linden M, Ansari T, Jeffery P K, Persson C G A. Allergen-induced eosinophil cytolysis is a primary mechanism for granule protein release in human upper airways. Am. J. Respir. Crit. Care Med. 160: 304-312, 1999.
5. Leiferman K M. Eosinophils in atopic dermatitis. Allergy 44: 20-26, 1989.
6. Gleich G J, Motojima S, Frigas E, Kephart G M, Fujisawa T, Kravis L P. The eosinophilic leukocyte and the pathology of fatal bronchial asthma: evidence for pathologic heterogeneity. J. Allergy Clin. Immunol. 80: 412-415, 1987.
7. Suissa S, Ernst P. Inhaled corticosteroids: Impact on asthma morbidity and mortality. J. Allergy Clin. Immunol. 107: 937-944, 2001.
8. Macdessi J S, Randell T L, Donaghue K C, Ambler G R, van Asperen P P, Mellis C M. Adrenal crises in children treated with high-dose inhaled corticosteroids for asthma. Med. J. Aust. 178: 214-216, 2003.
9. Milgrom H, Berger W, Nayak A, Gupta N, Pollard S, McAlary M, Taylor A F, Rohane P. Treatment of childhood asthma with anti-immunoglobulin E antibody (omalizumab). Pediatrics 108: E36, 2001.
10. Frew A J, Plummeridge M J. Alternative agents in asthma. J. Allergy Clin. Immunol. 108: 3-10, 2001.
11. Niven A S, Argyros G. Alternate Treatments in Asthma. Chest 123:1254-1265, 2003.
12. Thomas P S, Geddes D M, Barnes P J. Pseudo-steroid resistant asthma. Thorax 54: 352-356, 1999.
13. Hodge L, Salome C M, Peat J K, Haby M M, Xuan W, Woolcock A J. Consumption of oily fish and childhood asthma risk. Med. J. Aust. 164: 137-40, 1996.
14. Spector S L, Surette M E. Diet and asthma: has the role of dietary lipids been overlooked in the management of asthma? Ann. Allergy Asthma Immunol. 90: 371-377, 2003.
15. Nagakura T, Matsuda S, Shichijyo K, Sugimoto H, Hata K. Dietary supplementation with fish oil rich in ω-3 polyunsaturated fatty acids in children with bronchial asthma. Eur. Respir. J. 16: 861-865, 2000.
16. Emelyanov A, Fedoseev G, Krasnoschekova 0, Abulimity A, Trendeleva T, Barnes P J. Treatment of asthma with lipid extract of New Zealand green-lipped mussel: a randomised clinical trial. Eur. Respir. J. 20: 596-600, 2002.
17. Woods R K, Thien F C, Abramson M J. Dietary marine fatty acids (fish oil) for asthma in adults and children. Cochrane Database Syst. Rev. CD001283, 2002
18. Wijga A H, Smit H A, Kerkhof M, de Jongste J C, Gerritsen J, Neijens H J, Boshuizen H C, Brunekreef B. Association of consumption of products containing milk fat with reduced asthma risk in pre-school children: the PIAMA birth cohort study. Thorax 58: 567-572, 2003.
19. Bolte G, Frye C, Hoelscher B, Meyer I, Wjst M, Heinrich J. Margarine consumption and allergy in children. Am. J. Respir. Crit. Care Med. 163: 277-279, 2001.
20. Dunder T, Kuikka L, Turtinen J, Rasanen L, Uhari M. Diet, serum fatty acids and atopic diseases in childhood. Allergy 56: 425-428, 2001.
21. von Mutius E, Weiland S K, Fritzsch C, Duhme H, Keil U. Increasing prevalence of hayfever and atopy among children in Leipzig, East Germany. Lancet 351: 862-866, 1998.
22. Haby M M, Peat J K, Marks G B, Woolcock A J, Leeder S R. Asthma in preschool children: prevalence and risk factors. Thorax 56: 589-595, 2001.
23. Parodi P W. Health benefits of conjugated linoleic acid. Food Industry J. 3: 222-259, 2002.
24. Whigham L D, Cook E B, Stahl J L, Saban R, Bjorling D E, Pariza M W, Cook M E. CLA reduces antigen-induced histamine and PGE2 release from sensitized guinea pig tracheae. Am. J. Physiol. Regulatory Integrative Comp. Physiol. 280: R908-R912, 2001.
25. Whigham L D, Higbee A, Bjorling D E, Park Y, Pariza M W, Cook M E. Decreased antigen-induced eicosanoid release in conjugated linoleic acid-fed guinea pigs. Am. J. Physiol. Regulatory Integrative Comp. Physiol. 282: R1104-R1112, 2002.
26. Parodi P W. Conjugated octadecadienoic acids of milk fat. J. Dairy Sci. 60: 1550-1553, 1977.
27. Bauman D E. Conjugated linoleic acid (CLA) and milk fat: A Good News Story. Proceedings of the 1st Annual Arizona Dairy Production Conference, Tempe, Ariz., Oct. 17, 2002 (http://animal.cals.arizona.edu/azdp/papers/2002/bauman.pdf).
28. Kelley D S, Erickson K L. Modulation of body composition and immune function by conjugated linoleic acid in humans and animal models: benefits vs. risks. Lipids 38: 377-386, 2003.
29. Clement L, Poirier H, Niot I, Bocher V, Guerre-Millo M, Krief S, Staels B, Besnard P. Dietary trans-10,cis-12 conjugated linoleic acid induces hyperinsulinemia and fatty liver in the mouse. J. Lipid Res. 43: 1400-1409, 2002.
30. Riserus U, Basu S, Jovinge S, Fredikson G N, Arnlov J, Vessby B. Supplementation with conjugated linoleic acid causes isomer-dependent oxidative stress and elevated C-reactive protein. A potential link to fatty acid-induced insulin resistance. Circulation 106: 1925-1929, 2002.
31. Chin, S. F., Liu, W., Storkson, J. M., Ha, Y. L. and Pariza, M. W. Dietary sources of conjugated dienoic isomers of linoleic acid, a newly recognised class of anticarcinogens, Journal of Food composition and Analysis, 5, 185-197, 1992.
32. Harfoot, C. G., and Hazlewood, G. P., “Lipid metabolism in the rumen” in P. N. Hobson (Ed.) “The Rumen Microbial Ecosystem” at pages 285 to 322, Elsevier Applied Science Publishers, London (1988).
33. Reeves P. G., Nielsen F. H., Fahey G. C., Jr, AIN-93 purified diets for laboratory rodents: final report of the American Institute of Nutrition ad hoc writing committee on the reformulation of the AIN-76A rodent diet. J. Nutr. 1993;123:1939-1951.
34. Luna L G, editor. Manual of histologic staining methods of the Armed Forces Institute of Pathology. New York: McGraw-Hill; 1968.
35. Keramidaris E, Merson T D, Steeber D A, Tedder T F, Tang M L. L-selectin and intercellular adhesion molecule 1 mediate lymphocyte migration to the inflamed airway/lung during an allergic inflammatory response in an animal model of asthma. J. Allergy Clin. Immunol. 107: 734-738, 2001.
36. Hartert T V, Dworski R T, Mellen B G, Oates J A, Murray J J, Sheller J R. Prostaglandin E(2) decreases allergen-stimulated release of prostaglandin D(2) in airways of subjects with asthma. Am. J. Respir. Crit. Care Med. 162: 637-40, 2000.
37. Trifilieff A, El-Hashim A, Bertrand C. Time course of inflammatory and remodeling events in a murine model of asthma: effect of steroid treatment. Am. J. Physiol. Lung Cell Mol. Physiol. 279: L1120-L1128, 2000.
38. Voynow J A., Fischer B M, Malarkey D E, Burch L H, Wong T, Longphre M, Ho S B, and Foster W M. Neutrophil elastase induces mucus cell metaplasia in mouse lung. Am. J. Physiol. Lung Cell Mol. Physiol. 287: L1293-L1302, 2004.
39. Stumptner C, Fuchsbichler A, Heid H, Zatlouka K, Denk H. Mallory body—A disease-associated type of sequestosome. Hepatol. 35:1053-1062, 2002.

1.-23. (canceled)

24. A pharmaceutical composition comprising milk fat enriched with c-9, t-11 CLA or a salt, ester or precursor thereof and a pharmaceutically acceptable carrier.

25. A pharmaceutical composition as claimed in claim 24 for treating or preventing a condition associated with one or more of leukocyte infiltration, eosinophilia, IgE secretion, airway remodeling, bronchoconstriction and mucus hypersecretion.

26. A pharmaceutical composition as claimed in claim 24 wherein the milk fat comprises at least about 2, 4, 6, 8, 10, 15, 20, 25, 30, 35, 40, 45, or 50% by weight of c-9, t-11, CLA or a salt, ester or precursor thereof.

27. A pharmaceutical composition as claimed in claim 24 wherein the milk fat comprises CLA isomers which comprise at least about 50, 55, 60, 65, 70, 75, 80, 85, 90, 95 or 99% c-9, t-11 CLA by weight.

28. A pharmaceutical composition as claimed in claim 24 wherein the c-9, t-11 CLA is selected from c-9, t-11 CLA derived from a natural source; synthetic c-9, t-11 CLA; c-9, t-11 CLA in free fatty acid form; c-9, t-11 CLA bound to glycerol, a monoglyceride or a diglyceride; c-9, t-11 CLA in esterified form; or mixtures thereof.

29. A pharmaceutical composition as claimed in claim 24 wherein the c-9, t-11 CLA precursor is vaccenic acid.

30. A pharmaceutical composition as claimed in claim 24 wherein the composition is substantially free of t-10, c-12 CLA, the milk fat is substantially free of t-10, c-12 CLA or the milk fat otherwise has a fatty acid profile that corresponds substantially to the fatty acid profile of normal milk fat.

31. A pharmaceutical composition as claimed in claim 24 wherein the milk fat is produced by feeding a milk producing mammal with a diet enriched with at least one fatty acid or by combining a source of c-9, t-11 CLA or a salt, ester or precursor thereof with milk fat.

32. A pharmaceutical composition as claimed in claim 24 which is formulated for oral, nasal, topical, subcutaneous, intramuscular or intravenous administration.

33. A pharmaceutical composition as claimed in claim 24 which is formulated for ingestion, inhalation or topical application.

34. A pharmaceutical composition as claimed in claim 24 which is formulated as an inhalable powder, inhalable solution or aerosol.

35. A method of treating or preventing a condition associated with one or more of leukocyte infiltration, eosinophilia, airway remodeling and bronchoconstriction comprising administering c-9, t-11 CLA or a salt, ester or precursor thereof to a subject in need thereof.

36. A method of treating or preventing a condition associated with one or more of leukocyte infiltration, eosinophilia, IgE secretion, airway remodeling, bronchoconstriction and mucus hypersecretion comprising administering milk fat enriched with c-9, t-11 CLA or a salt, ester or precursor thereof to a subject in need thereof.

37. A method as claimed in claim 36 wherein the milk fat comprises at least about 2, 4, 6, 8, 10, 15, 20, 25, 30, 35, 40, 45, or 50% by weight of c-9, t-11 CLA or a salt, ester or precursor thereof.

38. A method as claimed in claim 36 wherein the milk fat comprises CLA isomers which comprise at least about 50, 55, 60, 65, 70, 75, 80, 85, 90, 95 or 99% c-9, t-11 CLA by weight.

39. A method as claimed in claim 36 wherein the c-9, t-11 CLA is selected from c-9, t-11 CLA derived from a natural source; synthetic c-9, t-11 CLA; c-9, t-11 CLA in free fatty acid form; c-9, t-11 CLA bound to glycerol, a monoglyceride or a diglyceride; c-9, t-11 CLA in esterified form; or mixtures thereof.

40. A method as claimed in claim 36 wherein the c-9, t-11 CLA precursor is vaccenic acid.

41. A method as claimed in claim 36 wherein substantially no t-10, c-12 CLA is administered to the subject.

42. A method as claimed in claim 36 wherein the milk fat is substantially free of t-10, c-12 CLA or wherein the milk fat otherwise has a fatty acid profile that corresponds substantially to the fatty acid profile of normal milk fat.

43. A method as claimed in claim 36 wherein the milk fat is produced by enhancing natural levels of CLA in milk by feeding a milk producing mammal with a diet enriched with at least one fatty acid or the milk fat is prepared by combining a source of c-9, t-11 CLA or a salt, ester or precursor thereof with milk fat.

44. A method as claimed in claim 36 wherein the milk fat is formulated for oral, nasal, topical, subcutaneous, intramuscular or intravenous administration.

45. A method as claimed in claim 36 wherein the milk fat is formulated for ingestion, inhalation or topical application.

46. A method as claimed in claim 36 wherein the milk fat is formulated as an inhalable powder, inhalable solution or aerosol.

47. A method as claimed in claim 36 wherein the condition is asthma.

48. A method as claimed in claim 36 wherein the condition is an atopic condition.

49. A method as claimed in claim 36 wherein the condition is selected from allergic rhinitis, hay fever, atopic rhinoconjunctivitis, urticaria, asthma and atopic eczema.

50. A method as claimed in claim 36 wherein the condition is an allergy.

51. A method as claimed in claim 36 wherein the condition is selected from contact dermatitis, eczema, hives (urticaria), allergic conjunctivitis, hay fever, allergic rhinitis, airborne allergies including tree, weed, and grass pollen allergies, latex allergies, food allergies including peanut, shellfish and milk protein allergies, drug allergies, insect sting allergies including honeybee allergies, wasp allergies, hornet allergies, yellow jacket allergies, fire ant allergies, mold allergies including allergies to alternaria, cladosporium, aspergillus, penicillium, helminthosporium, epicoccum, fusarium, mucor, rhizopus, and auerobasidium, dust mite allergies, animal allergies, allergic bronchopulmonary aspergillosis, occupational asthma, and episodic angioedema with eosinophilia.

52. A method as claimed in claim 36 wherein the condition is an eosinophilia.

53. A method as claimed in claim 36 wherein the condition is selected from airway, lung, blood and skin eosinophilia, eosinophilic ascites, eosinophilic cellulitis, eosinophilic fascitis, eosinophilic gastroenteritis, coeliac disease, allergic colitis, eosinophilic esophagitis, eosinophilic pancreatitis, eosinophilic pneumonias, bronchiectasis, eosinophilic synovitis, nasal eosinophilia, tropical pulmonary eosinophilia, Churg Strauss syndrome, pulmonary eosinophilia, idiopathic hypereosinophilic syndrome, inflammatory bowel disease, eosinophilic cholangitis, eosinophilic leukaemia and other eosinophilic cancers, familiar (hereditary) eosinophilia, eosinophilic granuloma, sarcoidosis, eosinophilia-myalgia syndrome, cystic fibrosis, nasal polyposis, eosinophil meningitis, Wegener's granulomatosis, polyarteritis nodosa, rheumatoid arthritis, pemphigus vulgaris, bullous pemphigoid, dermatitis herpetiformis, erythema multiforme, eosinophilic cellulites, and parasitic infections including Ascaris Toxocara canis, Filariasis, Anchylostomiasis, Trichinosis, Strongvloidiasis, Fascioliasis, and Schistosomiasis.

54. A method as claimed in claim 36 wherein the condition is a Th2 mediated condition.

55. A method as claimed in claim 36 wherein the condition is selected from Th2 mediated asthma, allergies, eczema, microbial or parasite infection, and autoimmune diseases including ulcerative colitis.

56. A method for treating or preventing a condition associated with one or more of leukocyte infiltration, eosinophilia, IgE secretion, airway remodeling, bronchoconstriction and mucus hypersecretion with steroid sparing effect comprising administering c-9, t-11 CLA or a salt, ester or precursor thereof or milk fat enriched with c-9, t-11 CLA or a salt, ester or precursor thereof to a subject in need thereof.

57. A method as claimed in claim 56 wherein the condition is a steroid-dependent condition including corticosteroid dependent asthma, severe eczema and eosinophilic disorders including eosinophilic gastroenteritis, eosinophilic pneumonia and hypereosinophilic syndrome.
